(19) United States
(12) Patent Application Publication
(10) Pub. No.: US 2012/0189687 A1
(43) Pub. Date: Jul. 26, 2012

(54) Compositions and Methods for Topical Application of Growth Factors and Cytokines

(76) Inventor: Benjamin Johnson, Evergreen, CO (US)

(21) Appl. No.: 12/978,694

(22) Filed: Dec. 27, 2010

(51) Int. Cl. A61K 9/127; A61P 9/00; A61K 38/18; A61K 38/19; A61P 37/04; A61P 17/00

(52) U.S. Cl. 424/450; 514/7.6

(57) ABSTRACT

A composition for therapeutically enhancing skin health by allowing growth factors derived from stem cells or fibroblasts to penetrate within the epidermis to increase the proliferation, growth and differentiation of skin cells. The composition includes at least one and preferably plural growth factors that are encapsulated within liposomes. Cytokines may also be encapsulated as well. The liposomally encapsulated growth factors and/or cytokines are able to penetrate into the basal skin cell layer to attach to the skin cells or penetrate through the follicles to reach the dermis.

### FIELD OF THE INVENTION

This invention relates to the field of applying growth factors and cytokines to the skin, and particularly for applying growth factors and cytokines derived from stem cells and fibroblasts.

### BACKGROUND OF THE INVENTION

The human skin undergoes constant cellular replacement. Millions of new skin cells are generated daily through cell reproduction and division. These new cells are generated in the basal cell layer at the deepest layer of the epidermis by proliferation, growth and differentiation and migrate upwards to the outer layer of the epidermis. New cells are also formed in the dermis where much of wound healing actually occurs. This production of skin cells is critical in maintaining proper skin health and the ability of the body to heal from wounds and infections.

The human body naturally makes proteins referred to as human growth factors and cytokines that bind to receptors on the cell surface to activate cellular proliferation and/or differentiation. Growth factors and cytokines are critical to the ability of the skin to produce the necessary skin cells for healthy skin. As the body ages, it produces fewer and fewer growth factors that can result in less healthy skin. Growth factors are also important in the ability of the body to heal from skin wounds. The availability of growth factors in the skin also combat aging effects such as increasing the production of collagen, elastin and glycosamoniglycans which are responsible for elasticity and structural integrity within the skin. Growth factors provide immune activity for scar revisions, to fight infection, to remove toxins and to heal pre-cancerous lesions. Growth factors also enhance new blood vessel formation to increase moisture and nutrition in the skin.

Another factor in the proliferation and differentiation of skin cells is the action of cell-signaling protein molecules referred to as cytokines. Cell growth, division, differentiation and functioning are affected by cytokines. The ability of the human body to generate cytokines is also decreased with age.

Thus, the ability of the skin to manufacture growth factors and cytokines is critical to maintain skin health. This ability significantly decreases as the body ages. There have been a number of efforts to supplement the production of growth factors and cytokines in the skin. Among these efforts is the use of topical application of growth factors and cytokines to the skin. These have limited success due to the size of the proteins and the inability for those proteins to migrate downward through the epidermis to the basal cell layer. Some penetration occurs through the hair follicles but this is also limited by the size of these growth factors.

### SUMMARY OF THE INVENTION

The present invention provides a skin care product that supplements the declining number of growth factors in aging skin thereby significantly improving skin health. The skin care product is able to deeply penetrate the epidermis of the skin with a topical application. Growth factors and/or cytokines penetrate more effectively with liposomal support thereby enhancing penetration to the deeper layers of the epidermis and upper epidermis which increases the proliferation, division and differentiation of the skin cells. The skin care product not only improves the health of the skin by increasing the number of skin cells being produced but also is able to provide anti-aging effects to the skin by increasing the production of collagen and elastin, and improving the immune activity of the skin for wounds, scar revisions, infections, and per-cancerous lesions.

The skin care product of a preferred embodiment includes at least one and preferably a plurality of growth factors derived from stem cells that are of particular applicability in maintaining the health of the skin. Cytokines may also be provided as well. The growth factors and/or cytokines are then encapsulated within a liposomal solution. The liposomally encapsulated growth factors and/or cytokines are then included within a topical skin application. The product is then applied topically to areas of the skin that has been damaged or suffers from poor health. The encapsulated growth factors and/or cytokines are able to penetrate the epidermis into the basal skin cell layer and upper dermis. The growth factors and/or cytokines attach to the skin cells to increase the proliferation, growth, division and differentiation of the skin cells.

These and other features of the present invention will be evident from the ensuing detailed description of preferred embodiments and from the drawings.

### DETAILED DESCRIPTION OF PREFERRED EMBODIMENTS

The present invention provides products and methods for maintaining the health of skin. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

In accordance with the present invention, human growth factors and/or cytokines are incorporated with liposomes in therapeutic compositions for topical application to prevent or alleviate the conditions and symptoms of cosmetic and dermatological disorders are described as follows. In particular, growth factors and/or cytokines that are derived from stem cells and combined with liposomes to increase the penetration of the growth factor proteins into the skin, and in particular to the basal cell layer and dermis to increase the proliferation, growth, division and differentiation of skin cells.

### Growth Factors

Growth factors and cytokines are very difficult to make and have primarily come from programming bacteria to produce these proteins. In complete contrast, growth factors and cytokines from stem cells and/or fibroblasts are made by human cells. The advantage of stem cell manufacturing is that the growth factors are much more stable and activate the skin cells better because of their 3-D structure. Manufactured growth factors have a much shorter shelf life and are typically more linear. While a few companies are making products with stem cell and/or fibroblast growth factors, the ability to enhance their delivery and performance has been lacking prior to the present invention.

Growth factors and cytokines derived from stem cells or fibroblasts are readily available from a number of commercial biotechnology companies. The growth factors of the preferred embodiment of the present invention include but are not limited to the following growth factors and cytokines:

| Cytokine/Growth factor | Pg/ml |
|---|---|
| TGF-β1 | 2010.0 |
| TGF-β2 | 733.0 |
| TGF-β3 | 82.0 |
| PDGF-AA | 1620.0 |
| PDGF-AB-BB | 128.25 |
| EGF | 28.0 |
| VEGF | 14.0 |
| PGF2 | 432.0 |
| MIP1a | 45.20 |
| GMC-SF | 109.7 |
| Flt3 | 10.0 |
| Interleukin-1a | 12.7 |
| Interleukin-1a | 2.0 |
| Interleukin-2 | 4.8 |
| Interleukin-3 | 26.0 |
| Interleukin-4 | 420.1 |
| Interleukin | 0.0 |
| Interleukin-6 | 427.0 |
| Interleukin-7 | 26.6 |
| Interleukin-8 | 420.1 |
| Interleukin-10 | 1.2 |
| Interleukin-12p40 | 19.3 |
| Interleukin-13 | 1.4 |
| Interleukin-15 | 167.7 |
| IFNg | 19.6 |
| TNFα | 16.4 |
| Eotaxin | 123.50 |
| MCP-1 | 756.0 |
| IP-10 | 12.4 |

Other growth factors and cytokines may be used as well.

### Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

### Preparation of Exemplary Therapeutic Compositions

Accordingly, a preferred embodiment of the present invention provides cosmetic as well as therapeutic compositions containing growth factors and/or cytokines coated in liposomal material which when topically administered will increase the proliferation, growth, division and differentiation of cells. These cells are then able to improve skin health as well as to treat skin wounds and infections.

The growth factors and cytokines of the instant invention may be formulated for topical application in aqueous or non-aqueous solution, gel, lotion, cream or ointment containing 0.1 to 20 percent and preferably from 0.5 to 15 percent by weight of the total composition. Other additives may be used to stabilize or otherwise provide functionality such as solvents, plasticizers, emulsify, stiffen, or other functions. Liposomal lecithin or a liposome substitute or other lipid preparations are added to the above solution with mixing until a uniform consistency is obtained.

To prepare a typical aqueous solution, the growth factors and/or cytokines are dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, the growth factors and/or ctyokines are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing growth factors and/or cytokines is prepared by first dissolving growth factors in ethanol, acetone, propylene glycol or other solvent. The solution thus prepared is then admixed with commonly available oil-in-water emulsions. BHT or sodium metabisulfite may be added to such emulsions to stabilize the growth factors. Liposomes or liposome substitutes are added to this solution with mixing until a uniform consistency is achieved.

A typical gel composition is formulated by first dissolving growth factors and/or cytokines in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

### Therapeutic Uses

The present invention may in various embodiments be used to increase the efficacy of the use of growth factors and cytokines for therapeutically and cosmetically treating many skin disorders.

A preferred embodiment of the present invention increases the efficacy of a topical skin care product by increasing the penetration of growth factors that have been derived from stem cells or fibroblasts into the skin. Compositions containing growth factors are coated or mixed with liposomal materials as described above. The liposomal growth factors compound has been shown to increase the penetration of the growth factors thereby increasing the efficacy of the growth factors product.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

What is claimed is:

1. A method for therapeutically enhancing skin health, said method comprising the steps of:
providing at least one growth factor derived from one of the group of: stem cells and fibroblasts;
encapsulating said at least one growth factors within a liposome;
topically applying said encapsulated at least one growth factor to a portion of the skin of an individual for absorption into the epidermis and dermis of the skin.

2. The method of claim 1 wherein said step of providing at least one growth factor includes:
providing at least one growth factor derived from stem cells.

3. The method of claim 1 wherein said step of providing at least one growth factor includes:
providing at least one growth factor derived from fibroblasts.

4. The method of claim 1 wherein said method further includes:
providing at least one cytokine derived from one of the group of: stem cells and fibroblasts;
encapsulating said at least one cytokines within a liposomes; and
including said encapsulated at least one cytokine in a topical application for absorption into the epidermis of the skin.

5. The method of claim 4 wherein said step of providing at least one cytokine includes:
providing at least one cytokine derived from stem cells.

6. The method of claim 4 wherein said step of providing at least one cytokine includes:
providing at least one cytokine derived from fibroblasts.

7. The method of claim 1 wherein said method includes:
increasing the production and differentiation of cells in the skin by the penetration of liposomally encapsulated growth factors into the epidermis and dermis.

8. The method of claim 1 wherein said method further includes:
increasing the tone and elasticity of the skin by the penetration of liposomally encapsulated growth factors into the epidermis by increasing the production of collagen and elastin.

9. The method of claim 1 wherein said method further includes:
increasing the moisturization of the skin by the penetration of liposomally encapsulated growth factors into the epidermis and dermis by increasing the production of glycosoaminoglycans and enhancing the epidermal barrier.

10. The method of claim 1 wherein said method further includes:
increasing the immune activity of the skin against scar revisions by the penetration of liposomally encapsulated growth factors in the epidermis and dermis.

11. The method of claim 1 wherein said method further includes:
increasing the immune activity of the skin to fight infection by the penetration of liposomally encapsulated growth factors in the epidermis and dermis.

12. The method of claim 1 wherein said method further includes:
increasing the immune activity of the skin to heal per-cancerous lesions by the penetration of liposomally encapsulated growth factors in the epidermis and dermis.

13. The method of claim 1 wherein said method further includes:
enhancing new blood vessel formation to provide moisture and nutrition for the skin by the penetration of liposomally encapsulated growth factors in the epidermis and dermis.

14. A skin care product for therapeutically enhancing skin health, said composition comprising:
at least one growth factor derived from one of the group consisting of: stem cells and fibroblasts; and
a liposome solution encapsulating said at least one growth factor.

15. The product of claim 14 wherein said product includes:
at least one growth factor derived from stem cells.

16. The skin care product of claim 14 wherein said product includes:
at least one growth factor derived from fibroblasts.

17. The skin care product of claim 14 wherein said composition further includes:
at least one cytokine derived from one of the group consisting of: stem cells and fibroblasts encapsulated within said liposomal solution.

18. The skin care product of claim 17 wherein said composition further includes:
at least one cytokine derived from stem cells.

19. The skin care product of claim 17 wherein said skin care product further includes:
at least one cytokine derived from fibroblasts.
